(19) United States
(12) Patent Application Publication
(10) Pub. No.: US 2026/0297644 A1
(43) Pub. Date: Oct. 1, 2026

(54) PEPTIDES PREPARED FROM A PLANT EXTRACT OBTAINED BY PROTEASE DIGESTION

(71) Applicant: Valogen Biosciences Limited, Kingsbridge (GB)

(72) Inventors: Harry Ross, Kingsbridge (GB); Raymond Sloan, York (GB)

(73) Assignee: Valogen Biosciences Limited, Kingsbridge (GB)

(21) Appl. No.: 19/479,639

(22) PCT Filed: May 8, 2024
(86) PCT No.: PCT/GB2024/051192

§ 371 Date: Oct. 28, 2025

(30) Foreign Application Priority Data
May 9, 2023 (GB) 2306822.4
Mar. 28, 2024 (GB) 2404497.6

(51) Int. Cl. C12P 21/06; A61K 8/64; A61K 36/31; A61K 38/01; A61K 45/06; A61P 17/02; A61Q 19/00

(52) U.S. Cl. CPC C12P 21/06; A61K 8/645; A61K 38/011; A61K 45/06; A61P 17/02; A61Q 19/00; A61K 36/31

(57) ABSTRACT

We disclose a process for the preparation of a plurality of plant derived peptides; compositions comprising said plurality of peptides; medicaments and cosmetics comprising said peptides and their use in the treatment of cutaneous skin conditions such as wound healing and inflammatory disease or conditions.

### FIELD OF THE DISCLOSURE

We disclose a process for the preparation of a plurality of plant derived peptides; compositions comprising said plurality of peptides; nutraceuticals, medicaments and cosmetics comprising said peptides; and their use in the treatment of skin conditions wound healing and inflammatory diseases and conditions.

### BACKGROUND TO THE DISCLOSURE

Oilseed rape (Brassica napus) is a cold-tolerant winter/spring crop cultivated primarily for the extraction of its vegetable oil (Raboanatahiry et al., 2021). Traditionally rapeseed oil was produced as a mechanical lubricant (Woźniak et al., 2019) and for other industrial purposes rather than for food production due to high levels of erucic acid, which is known to induce myocardial lipidosis in rodents (Kramer et al., 1992), and high levels of glucosinolates which gave the crop an unpalatable bitter taste. Consequently, Brassica napus has been selectively bred for food production by reducing the levels of erucic acid and glucosinolates (Woodfield and Harwood, 2017) and is commonly referred to as canola or LEAR (low erucic acid rape) oil (Russo et al., 2021). Today Brassica napus is a significant oil crop generating around 13% of the world's edible oil (Lu et al., 2011) with 79.5 million tonnes predicted to be harvested globally in 2022/2023 (European Commission, 2022).

Protein hydrolysates containing bioactive peptides are typically produced through the enzymatic hydrolysis or gastrointestinal digestion of animal or plant proteins (Chakrabarti et al., 2018). Bioactive peptides are low molecular weight peptides generally with an amino acid chain length of 2-20 amino acids and are thought to exert several physiological functions beneficial to human health, such as immunomodulatory, antihypertensive, antimicrobial or antioxidative properties (Zaky et al., 2022). The bioavailability of bioactive peptides offers a unique avenue for enhancing human health. For instance, peptides can penetrate deeper into certain tissue than their relatively cumbersome protein parents (Williams et al., 2015), which may be of benefit to the field of nutraceuticals or natural cosmetics. The therapeutic potential of bioactive rapeseed peptides noted in recent studies provides increasingly promising results and a mandate for additional research that may promote such entities as active agents in products such as anti-ageing creams or chemotherapeutics. For example, a recent study investigating the antiproliferative effects of rapeseed protein hydrolysates on hormone-dependent breast cancer (MCF-7) cells found that rapeseed proteins undergoing 8.5-9.0% hydrolysis with an average mass of 10 kDa suppresses MCF-7 (83.9%) proliferation whilst not impairing human fibroblast viability (Ferrero et al., 2021). The selective bioactive impact on cancerous/diseased cells is associated with the molecular weight, which, when considered with overall charge and hydrophobicity, seems to be the crucial elements impacting the bioavailability of peptides (Amigo and Hernández-Ledesma, 2020). Further research is required to delineate the association between factors that govern rapeseed hydrolysate bioavailability and penetration/delivery efficiency of cosmetics and therapeutics and, more broadly, to define the effects of such peptides on healthy human epidermal keratinocytes.

Although others have discovered that many plant-based hydrolysates can reduce radical oxygen species, increase levels of antioxidants (Tadesse and Emire, 2020), and exert a beneficial impact to the skin (e.g., promoting wound healing) (Song et al., 2019), to date, there is little evidence detailing the effects of rapeseed protein hydrolysates on the epidermis. To this end, in phase one of the study, we aim to provide insight into the compositional heterogeneity of rapeseed bioactive peptides and explore the consequence of increasing concentrations of such peptides to human epidermal keratinocyte proliferative capabilities to provide a biological rationale for the potential use in epidermal therapeutics, inflammatory diseases and conditions, nutrition and cosmetics.

The disclosure relates to a plurality of plant derived peptides and their use in medicaments, particularly but not exclusively with application in wound healing, anti-aging, anti-inflammatory disease and conditions and cosmetic enhancement of skin.

### STATEMENTS OF INVENTION

The disclosure relates to a composition comprising a plurality of peptides wherein said peptides are prepared from a plant extract obtained by protease digestion.

According to an aspect of the invention there is provided a composition comprising a plurality of peptides wherein said peptides are prepared from a plant extract obtained by protease digestion.

According to an aspect of the invention there is provided a composition comprising a plurality of peptides wherein said peptides are prepared from a plant extract obtained by protease digestion wherein said plurality of peptides is for use as a medicament.

According to an alternative aspect of the invention there is provided a composition comprising a plurality of peptides wherein said peptides are prepared from a plant extract obtained by protease digestion wherein said plurality of peptides is for use as a nutraceutical.

“Plurality” is construed as a composition comprising at least 2 peptides; preferably at least 3, 4, 5, 6, 7, 8, 9, or 10 peptides. Alternatively, plurality is construed as 10-15, 15 to 20 peptides, 20-25 peptides, 30-35 peptides, 35-40 peptides 40-45 peptides, 45-50 peptides, 50-55 peptides, 55-60 peptides, 60-65 peptides or 65 to 70 peptides. In a further alternative embodiment of the invention said plurality is construed as a composition comprising at least 70 peptides.

In a preferred embodiment of the invention said plurality of peptides comprise peptides that are at least 2 amino acids in length.

In a preferred embodiment of the invention said plurality of peptides comprise peptides that vary in length of between 4 to 25 amino acids.

In a preferred embodiment of the invention said plurality of peptides comprise peptides that vary in length of between 4 to 20 amino acids.

In a preferred embodiment of the invention said plurality of peptides comprise peptides that vary in length of between 4 to 18 amino acids.

In a preferred embodiment of the invention said plurality of peptides comprise peptides that vary in length of between 4 to 16 amino acids.

In a preferred embodiment of the invention said plurality of peptides comprise peptides that vary in length of between 4 to 14 amino acids.

In a preferred embodiment of the invention said plurality of peptides comprise peptides that vary in length of between 4 to 12 amino acids.

In a preferred embodiment of the invention said plurality of peptides comprise peptides that vary in length of between 8 to 12 amino acids.

In a preferred embodiment of the invention said plurality of peptides comprise peptides that vary in length of between 10 to 12 amino acids.

In a preferred embodiment of the invention said plant hydrolysate is obtained from a plant of the genus Brassica, for example Brassica napus or Canola.

In a preferred embodiment of the invention said plant hydrolysate is obtained from Canola.

In a preferred embodiment of the invention said hydrolysate is obtained by digestion with at least two endopeptidases. Preferably, at least one endopeptidase is subtilisin;

alternatively said second endopeptidase is bacillolysin; preferably said at least two endopeptidases are subtilisin and bacillolysin.

In a preferred embodiment of the invention said protease is Flavourzyme®

Preferred enzymes are subtilisin and bacillolysin. Alcalase® is a commercial preparation of subtilisin isolated from Bacillus subtilis. Protamex® is a commercial preparation of a complex consisting of subtilisin and a neutral protease called bacillolysin; see https://www.brenda-enzymes.org/enzyme.php?ecno=3.4.24.28. Flavourzyme® is a predominantly exoprotease with aminopeptidase activity and is isolated from Aspergillus oryzae.

In an alternative embodiment of the invention said composition comprises actinidin.

In a preferred embodiment of the invention actinidin is combined with at least one further protease.

Preferably, said further protease is substilisin.

In a preferred embodiment of the invention said further protease is bacillolysin

In a preferred embodiment said further protease includes both substilisin and of the invention bacillolysin.

In a preferred embodiment of the invention said nutraceutical comprises traces of sodium or sodium salts.

According to a further aspect of the invention there is provided a pharmaceutical composition comprising a plurality of peptides wherein said peptides are isolated from a plant hydrolysate obtained by protease digestion and including at least one excipient and/or carrier.

In a preferred embodiment of the invention there is provided a medicament or composition according to the invention wherein said medicament or composition further comprises a second different therapeutic agent.

In a preferred embodiment of the invention said second therapeutic agent is an analgesic, for example non-steroidal anti-inflammatory drugs.

According to an aspect of the invention there is provided a medicament or composition according to the invention for use in the treatment of a cutaneous or sub-cutaneous disease or condition.

In a preferred embodiment of the invention said condition is an ulcer, for example a cutaneous ulcer. For example, a diabetic ulcer associated with diabetic polyneuropathy.

In an alternative embodiment of the invention said cutaneous or sub-cutaneous disease or condition is selected from the group: acne, eczema, psoriasis and ichthyosis vulgaris.

According to an aspect of the invention there is provided a medicament or composition according to the invention for use in the treatment of a cutaneous wound.

In a preferred embodiment of the invention said cutaneous wound is a burn or scald.

According to an aspect of the invention there is provided a medicament or composition according to the invention for use as a cosmetic treatment.

In relation to cosmetic applications of the plurality of peptides its is envisaged that cosmetic applications are related to but not limited to anti-ageing (wrinkle reduction, antioxidants, stimulated collagen expression, reduction in production of metalloproteases, inhibition of glycation, stimulation of hyaluronic acid synthesis) balancing pigmentation (e.g., tyrosinase inhibition), humectants for hydration and similar applications, enhancement of hair growth and conditions that result in hair loss such as alopecia.

In a preferred embodiment of the invention said cosmetic treatment is hair conditioning.

Hair conditioning as known to the skilled artisan strengthens and hydrates the hair by providing a protective coating, prevents breakage, and/or promotes hair growth.

According to a further aspect of the invention there is provided a medicament or composition according to the invention for use in the treatment of hypertension.

In a preferred embodiment said composition comprises traces of sodium or sodium salts.

According to a further aspect of the invention there is provided a method for the preparation of a plurality of peptides comprising the steps:
i) preparation of plant material to provide an undigested plant homogenate;
ii) separation of the homogenate in i) into solid plant material and soluble plant material;
iii) treatment of said soluble plant material to provide a concentrated soluble plant extract; and
iv) digestion of said concentrated plant extract with one or more proteases, preferably endopeptidases.

Preferably, the homogenate of step i) comprises potassium hydroxide.

To maintain an alkaline pH, preferably at around pH 10-11, during the preparation of the homogenate strong bases such as NaOH or KOH are used. KOH is preferable as it results in preparations with trace amounts of sodium or sodium salts.

In a further preferred method of the invention the homogenate of step i) comprises traces of sodium or sodium salts.

According to an alternative aspect of the invention there is provided a method for the preparation of a plurality of peptides comprising the steps:
i) preparation of plant material to provide a plant homogenate combined with one or more proteases to form a combined plant/protease homogenate;
ii) incubation of the combined homogenate to form a protease digested homogenate; and optionally
iii) separating the protease digested plant homogenate from the undigested plant homogenate.

In a preferred method of the invention said plant material is a blend of plant meal and cake.

Preferably, the homogenate of step i) comprises potassium hydroxide.

In a further preferred method of the invention, the homogenate of step i) comprises traces of sodium or sodium salts.

Plant “meal” or “cake” are a by-product of the extraction process that extracts oil from plant material. Cake refers to the powder-like solid from for example, rapeseed which still contains an oil fraction. Meal is the same solid material but without the oil fraction as it has undergone further treatment (solvent extraction) to extract. Meal and cake are typically protein rich fractions.

In a preferred method of the invention said plant material the ratio of meal to cake is selected from the group: 10:90, 20:80, 30:70, 40:60, 50:50, 60:40, 70:30, 80:20 and 10:90.

Preferably said plant material is obtained from Brassica, preferably Brassica napus or Canola.

According to a further aspect of the invention there is provided a plurality of peptides obtained by the method according to the invention.

The medicaments/compositions according to the invention are preferably adapted for topical application in pharmaceutically acceptable preparations, for example as a cream or applied to a carrier such as a bandage. Such preparations may routinely contain pharmaceutically acceptable concentrations of salt, buffering agents, preservatives, and compatible carriers and also further agents such as collagen and/or collagen hydrolysates or peptides. Such amounts will depend, of course, on the condition being treated, the severity of the condition, the individual patient parameters including age, physical condition, size and weight, the duration of the treatment, the nature of concurrent therapy (if any), and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

The medicaments/compositions used contains an effective number of peptides for producing the desired response in a unit of weight or volume suitable for application to a patient. The medicaments/compositions applied to a subject can be chosen in accordance with different parameters, in particular the state of the subject, their body surface area, and their weight. Other factors include the desired period of treatment. If a response in a subject is insufficient at the initial doses applied, higher doses (or effectively higher doses by a different, more localized delivery route) may be employed to the extent that patient tolerance permits.

Administration of medicaments/compositions to mammals other than humans, (e.g., for testing purposes or veterinary therapeutic purposes), is carried out under substantially the same conditions as described above although dosages will vary in accordance with the size of the animal treated. A subject, as used herein, is a mammal, preferably a human, and including a non-human primate, cow, horse, pig, sheep, goat, dog, cat or rodent.

When applied to a subject the medicaments/compositions are provided in pharmaceutically acceptable amounts and in pharmaceutically acceptable compositions. The term “pharmaceutically acceptable” means a non-toxic material that does not interfere with the effectiveness of the biological activity of the active peptides. Such preparations may routinely contain salts, buffering agents, preservatives, compatible carriers, and optionally other therapeutic agents such as but not limited to, phytates, glucosinolates and kampferol. If topically applied the medicaments/compositions can optionally include penetration enhancers such as propylene glycol, transcutol, DMI, ethanol etc. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium, or calcium salts.

Medicaments/compositions according to the invention may be combined, if desired, with a pharmaceutically acceptable carrier. The term “pharmaceutically acceptable carrier” as used herein means one or more compatible solid or liquid fillers, diluents or encapsulating substances which are suitable for application to a human subject and are typically inert. The term “carrier” denotes an organic or inorganic ingredient, natural or synthetic, with which the active ingredient is combined to facilitate the application. The components of the medicaments also are capable of being co-mingled with the plurality of peptides, and with each other, in a manner such that there is no interaction which would substantially impair the desired pharmaceutical efficacy.

The medicaments/compositions are combined with pharmaceutically acceptable excipients, which may include: (a) fillers such as lactose, manitose, dicalcium phosphate, microcrystalline cellulose, starch, pre-gelatinised starch, (b) binders such as hydroxypropylmethyl cellulose, polyvinyl pyrrolidone, polyvinyl acetate, (c) powder flow enhancers such colloidal silicon dioxide (d) lubricants such as magnesium stearate, sodium stearyl fumarate (e) disintegrants such as sodium starch glycollate and polyvinyl pyrrolidone and (f) anti-sticking agents such as talc (g) taste masking agents such as sucrose, cellulose acetate, cellulose butyrate, polyvinyl acetate, polyvinyl alcohol, polymethacrylates.

According to an aspect of the invention there is provided a method to treat a subject comprising topically administering the medicament or composition according to the invention to a subject to treat a cutaneous disease or condition.

In a preferred method of the invention said cutaneous disease or condition is selected from the group: acne, eczema, psoriasis and ichthyosis vulgaris.

In a preferred method of the invention said condition is an ulcer, for example a cutaneous ulcer. For example, a diabetic ulcer associated with diabetic polyneuropathy.

According to an aspect of the invention there is provided a method to treat a subject comprising topically administering the medicament or composition according to the invention to a subject to treat a cutaneous wound.

In a preferred method of the invention said cutaneous wound is a burn or scald.

According to an aspect of the invention there is provided a method to cosmetically treat a subject in need of treatment comprising topically administering the medicament or composition according to the invention to the subject.

According to a further aspect of the invention there is provided a composition comprising a plurality of peptides wherein said peptides are prepared from a plant extract obtained from a plant of the genus Brassica by protease digestion obtained by digestion with at least two endopeptidases wherein said plurality of peptides is for use as a medicament.

According to a further aspect of the invention there is provided a composition comprising a plurality of peptides wherein said peptides are prepared from a plant extract obtained from a plant of the genus Brassica by protease digestion obtained by digestion with at least two endopeptidases wherein said plurality of peptides is for use as a nutraceutical.

In a preferred embodiment said nutraceutical comprises traces of sodium or sodium salts.

According to a further aspect of the invention there is provided a composition comprising a plurality of peptides wherein said peptides are prepared from a plant extract obtained from a plant of the genus Brassica by protease digestion obtained by digestion with at least two endopeptidases wherein said plurality of peptides is for use in the treatment of hypertension.

In a preferred embodiment said nutraceutical comprises traces of sodium or sodium salts.

The physiological requirement for sodium in humans is less than 1 g per day and the maximum daily intake of dietary sodium recommended by the World Health Organisation is 2 g or an equivalent of 5 g sodium salts (NaCl) per day for adults. NaCl is added to most food products to enhance the flavour. The composition of the application as filed provides a savoury taste and can therefore be used to enhance flavours of other food products without adding additional Na salts and is therefore suitable for the treatment of hypertension.

According to a further aspect of the invention there is provided a composition according to the invention for use in the treatment of an inflammatory disease or condition.

In a preferred embodiment of the invention said inflammatory disease or condition is an autoimmune inflammatory disease or condition.

In a preferred embodiment of the invention said inflammatory disease or condition is an inflammatory bowel disease.

In a preferred embodiment of the invention said inflammatory bowel disease is selected from the group consisting of: Crohn's disease, ulcerative colitis, coeliac disease collagenous colitis, lymphocytic colitis, ischaemic colitis, diversion colitis, Behcet's colitis.

In a preferred embodiment of the invention said inflammatory bowel disease is Crohn's disease.

In an alternative preferred embodiment of the invention said inflammatory bowel disease is ulcerative colitis.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

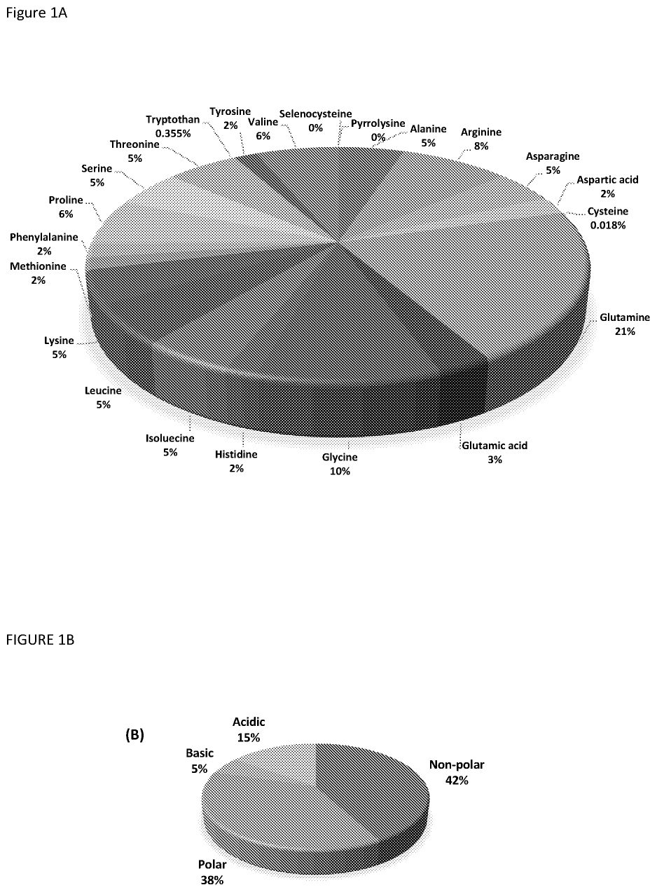

FIG. 1 Shown are pie charts representing mass spectrometry expression data from 995 identified filtrate 1 peptides illustrating the mean percentage composition according to exclusive amino acids (A) and by amino acid subtypes (acidic, basic, polar, and non-polar);

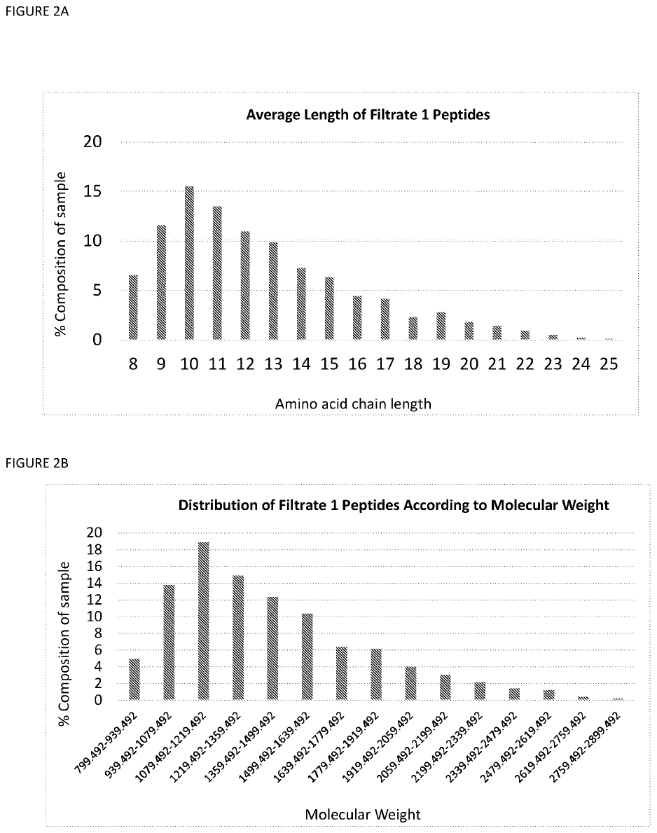

FIG. 2 Histogram depicting the amino acid chain length (A) and molecular weight distribution (B) of the 995 identified filtrate 1 peptides according to the percentage composition of the sample;

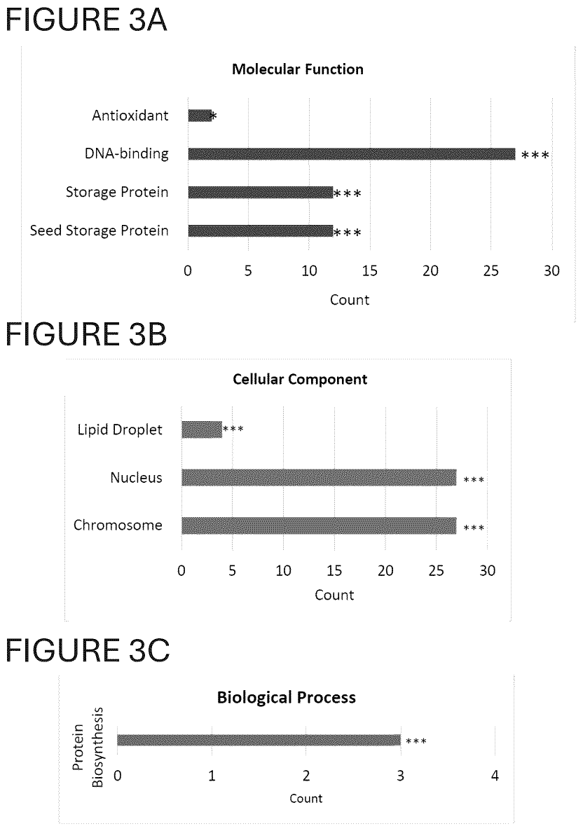

FIG. 3 Bar charts representing the results from a DAVID functional enrichment analysis of 73 filtrate 1 proteins. Gene ontology analysis classified according to the listed proteins known molecular function (A), cellular component (B), and biological process (C). The number of Asterix represents the strength of the p-value, 1 (P<0.05), 2 (P≤0.01), 3 (P<0.001);

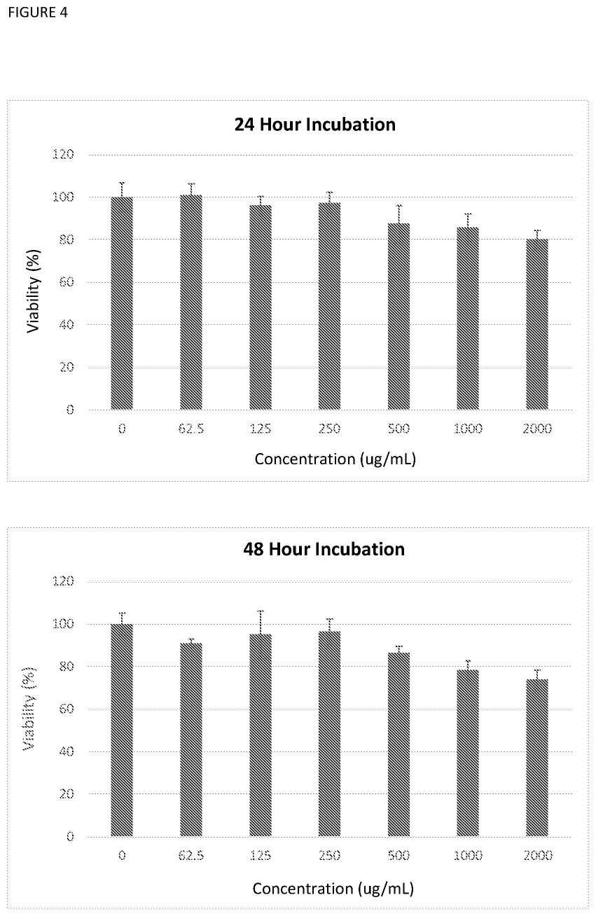

FIG. 4 Histogram plotting the results of 24- and 48-hour MTT proliferation assay conducted on HPEKp cells incubated with either no bioactive peptides or increasing concentrations of CnT-PR-dissolved filtrate 1 peptides ranging from 0-2000 ug/mL. Mean percentage viability values are plotted on the chart and calculated by comparing the absorbance of treated to untreated HPEKp cells. SEM is plotted;

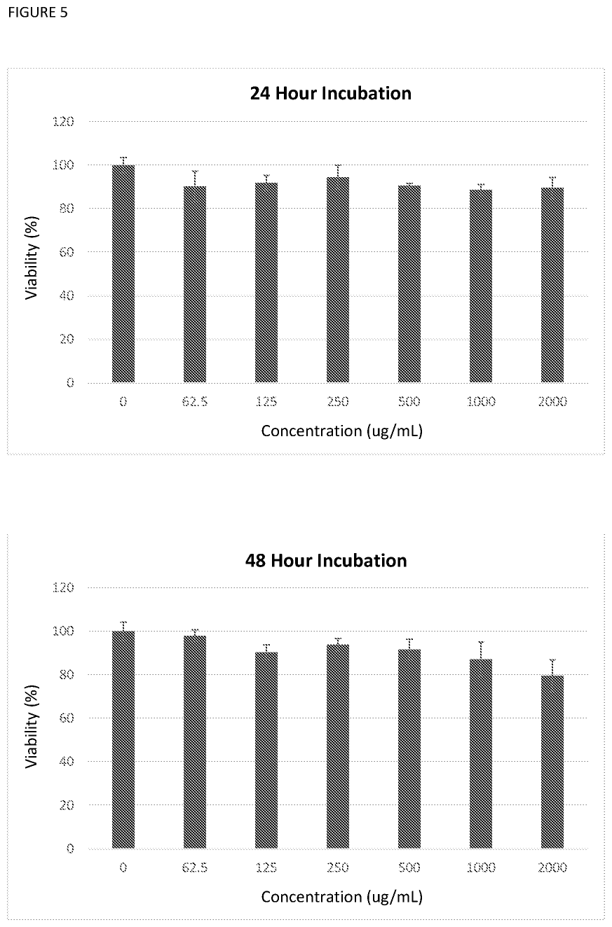

FIG. 5 Histogram plotting the results of 24- and 48-hour MTT proliferation assay conducted on human dermal fibroblast cells incubated with either no bioactive peptides or increasing concentrations of CNT-PR-F dissolved filtrate 1 peptides ranging from 0-2000 ug/ml. Mean percentage viability values are plotted on the chart and calculated by comparing the absorbance of treated to untreated HPEKp cells. SEM is plotted;

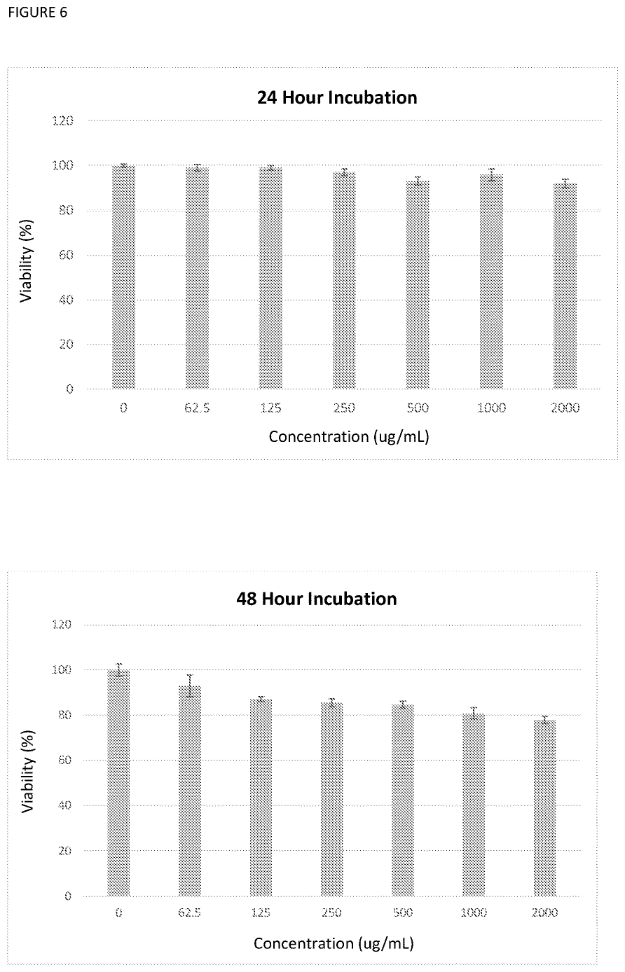

FIG. 6 Histogram plotting the results of 24- and 48-hour MTT proliferation assay conducted on A549 cells incubated with either no bioactive peptides or increasing concentrations of DMEM-dissolved filtrate 1 peptides ranging from 0-2000 ug/mL. Mean percentage viability values are plotted on the chart and calculated by comparing the absorbance of treated to untreated HPEKp cells. SEM is plotted;

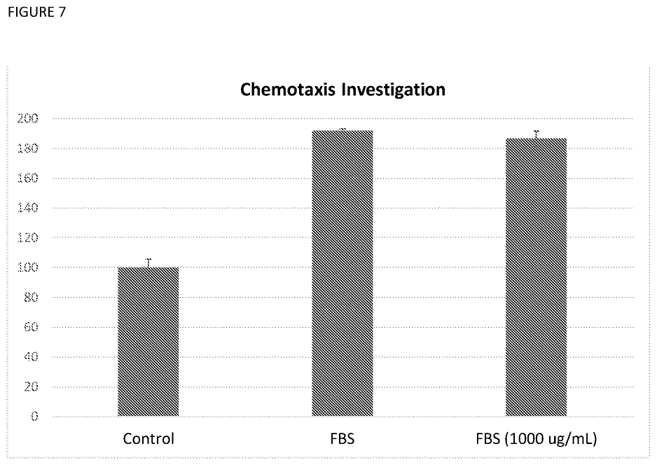

FIG. 7 Histogram representing the results of the chemotaxis assay involving HPEKp cells treated with or without 1000 μg/mL of filtrate 1 extracts in serum-based CNT-PR media. The percentage increase in migration over the course of 24 hours is compared to control untreated cells are plotted along with SEM;

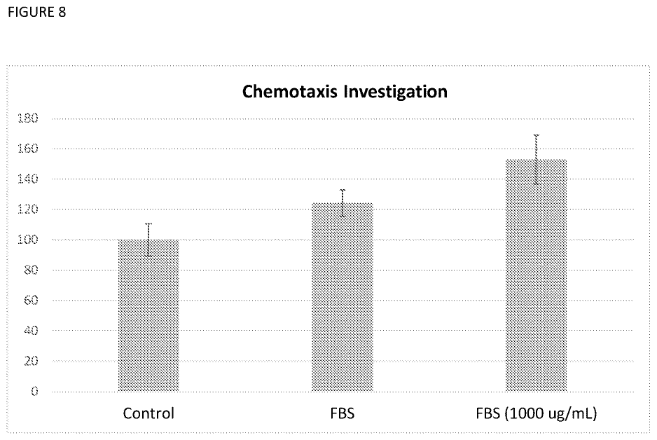

FIG. 8 Histogram representing the results of the chemotaxis assay involving HDF cells treated with or without 1000 μg/mL of filtrate 1 extracts in serum-based CNT-PR-F media. The percentage increase in migration over the course of 24 hours is compared to control untreated cells are plotted along with SEM;

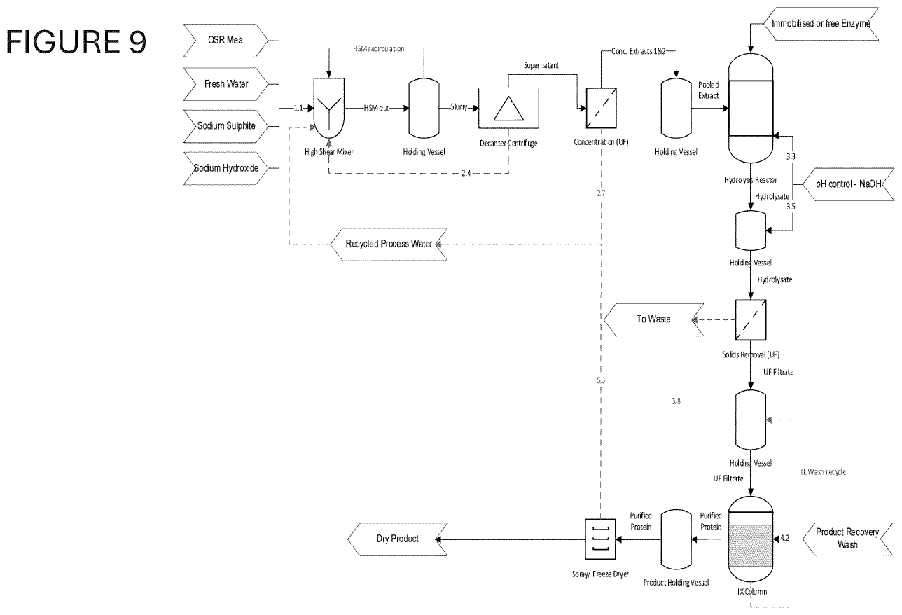

FIG. 9 schematic representation of the preparation of the protease hydrolysate according to the invention;

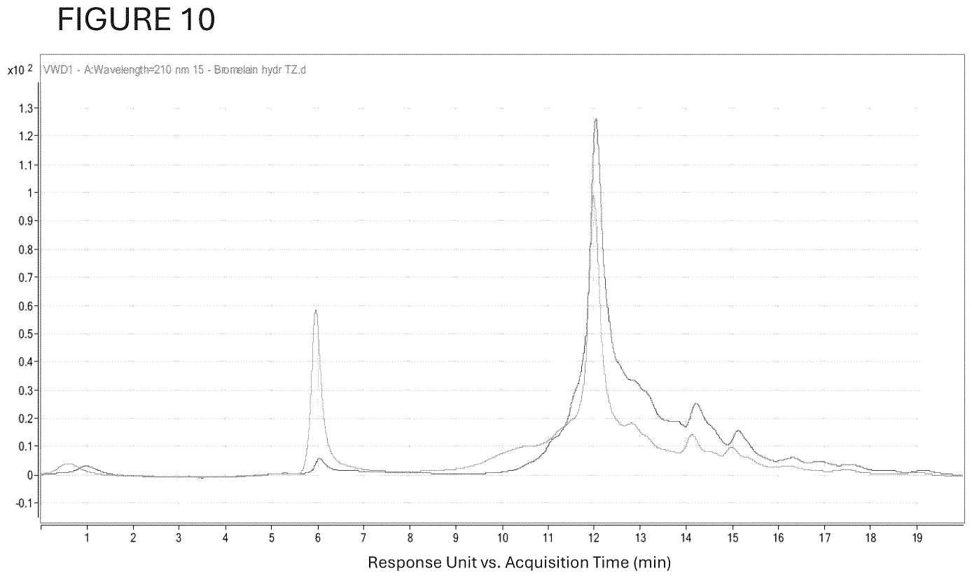

FIG. 10 illustrates hydrolysis carried out using a combination of Subtilisin and Protamex;

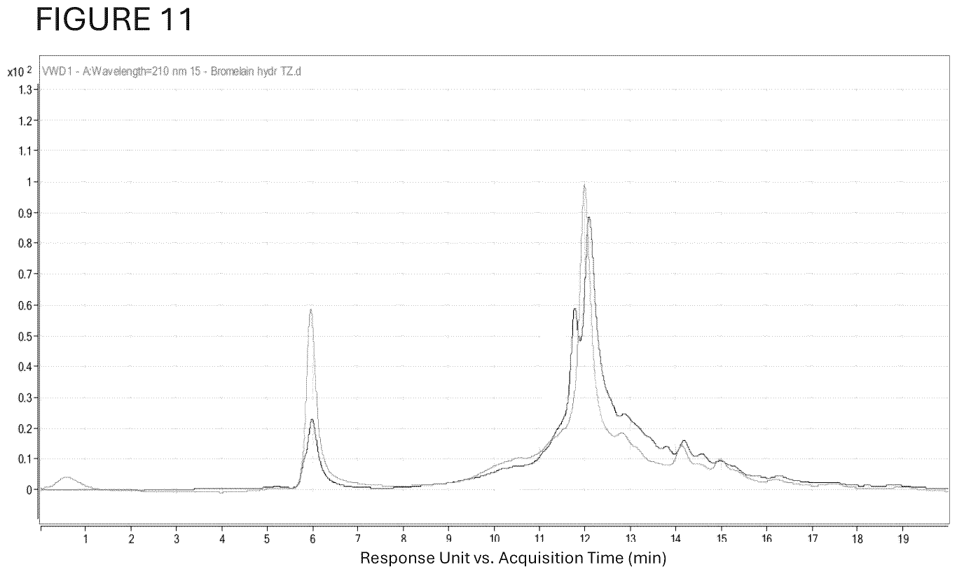

FIG. 11 illustrates hydrolysis carried out using actinidin;

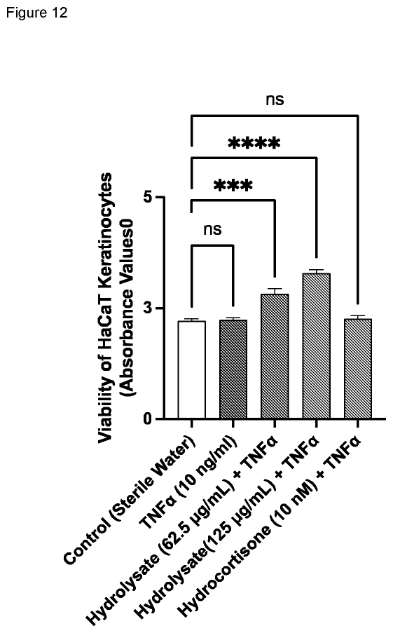

FIG. 12 shows the effects of hydrolysate (62.5 and 125 μg/mL) and hydrocortisone (10 nM) on the viability of HaCaT keratinocytes stimulated with recombinant human TNFα. Values are mean±SEM for 2 independent experiments. *** p<0.001, **** p<0.0001 compared with cells treated with sterile water only (control) using one-way ANOVA and post hoc Dunnett test for multiple comparisons;

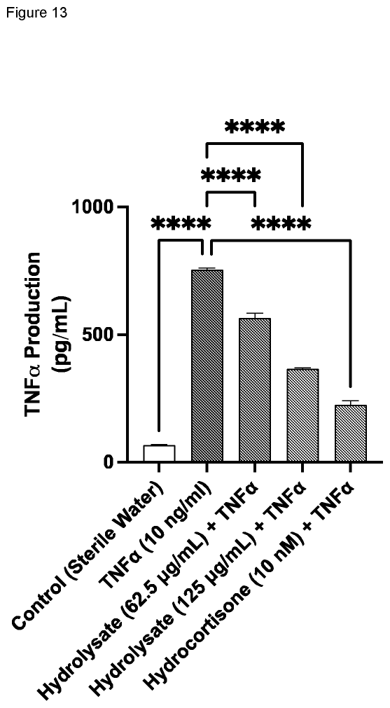

FIG. 13 shows the effects of hydrolysate (62.5 and 125 μg/mL) and hydrocortisone (10 nM) on elevated TNFα production in HaCaT keratinocytes stimulated with recombinant human TNFα. Values are mean±SEM for 2 independent experiments. **** p<0.0001 compared with TNFα-stimulated cells using one-way ANOVA and post hoc Dunnett test for multiple comparisons;

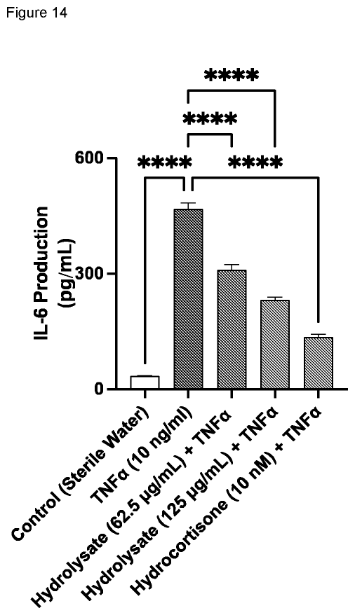

FIG. 14 shows the effects of hydrolysate (62.5 and 125 μg/mL) and hydrocortisone (10 nM) on elevated IL-6 production in HaCaT keratinocytes stimulated with recombinant human TNFα. Values are mean±SEM for 2 independent experiments. **** p<0.0001 compared with TNFα-stimulated cells using one-way ANOVA and post hoc Dunnett test for multiple comparisons;

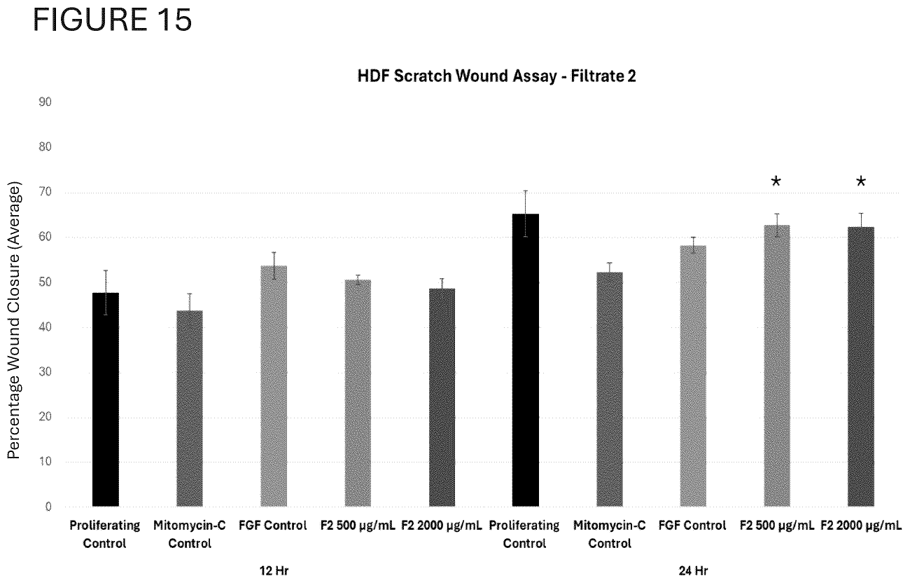

FIG. 15 is a histogram illustrating the outcomes of a scratch wound healing assay conducted on HDF. The experimental groups included: HDF untreated (proliferating control), treated with mitomycin-C only (mitomycin-C control), inhibited with mitomycin-C and treated with Fibroblast Growth Factor (FGF/positive control), or treated with mitomycin-C along with varying concentrations (500 μg/mL or 2000 μg/mL) of filtrate 2. Each experimental condition was performed in triplicates, with two measurements taken per replicate (on both left and right sides of the wound), resulting in a total sample size of n=6. The percentage wound area, determined using the ImageJ wound healing plugin, at the 0-hour timepoint served as the baseline for normalizing subsequent measurements at 12 and 24-hour timepoints for each experimental group. The data is represented as the mean percentage wound area with corresponding SEMs. A t-test assuming unequal variances was conducted, comparing each experimental group to the appropriate mitomycin-C control at a significance level of 5%. Significance levels are denoted by asterisks, with * indicating p<0.05, ** indicating p<0.01, and *** indicating p<0.001;

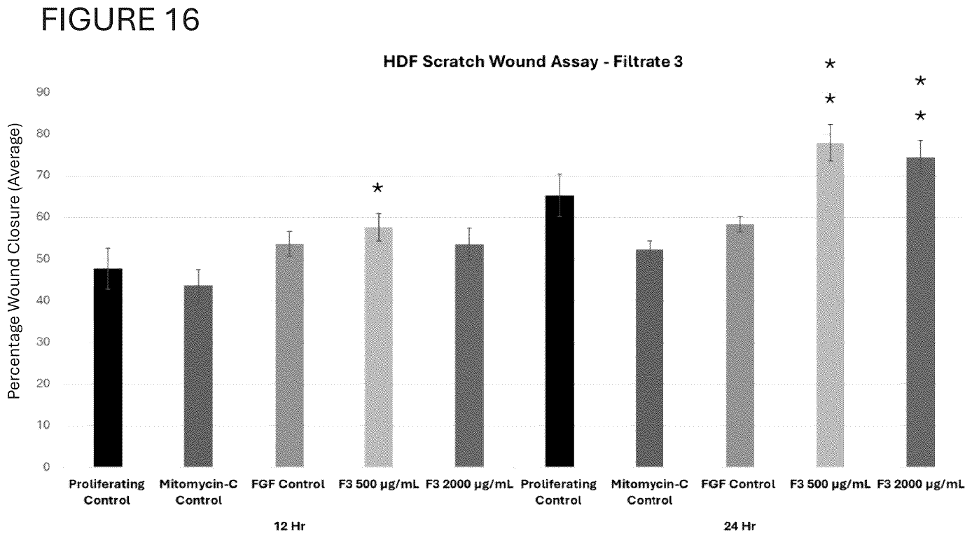

FIG. 16 is a histogram illustrating the outcomes of a scratch wound healing assay conducted on HDF. The experimental groups included: HDF untreated (proliferating control), treated with mitomycin-C only (mitomycin-C control), inhibited with mitomycin-C and treated with Fibroblast Growth Factor (FGF/positive control), or treated with mitomycin-C along with varying concentrations (500 μg/mL or 2000 μg/mL) of filtrate 3. Each experimental condition was performed in triplicates, with two measurements taken per replicate (on both left and right sides of the wound), resulting in a total sample size of n=6. The percentage wound area, determined using the ImageJ wound healing plugin, at the 0-hour timepoint served as the baseline for normalizing subsequent measurements at 12 and 24-hour timepoints for each experimental group. The data is represented as the mean percentage wound area with corresponding SEMs. A t-test assuming unequal variances was conducted, comparing each experimental group to the appropriate mitomycin-C control at a significance level of 5%. Significance levels are denoted by asterisks, with * indicating p<0.05, ** indicating p<0.01, and *** indicating p<0.001.

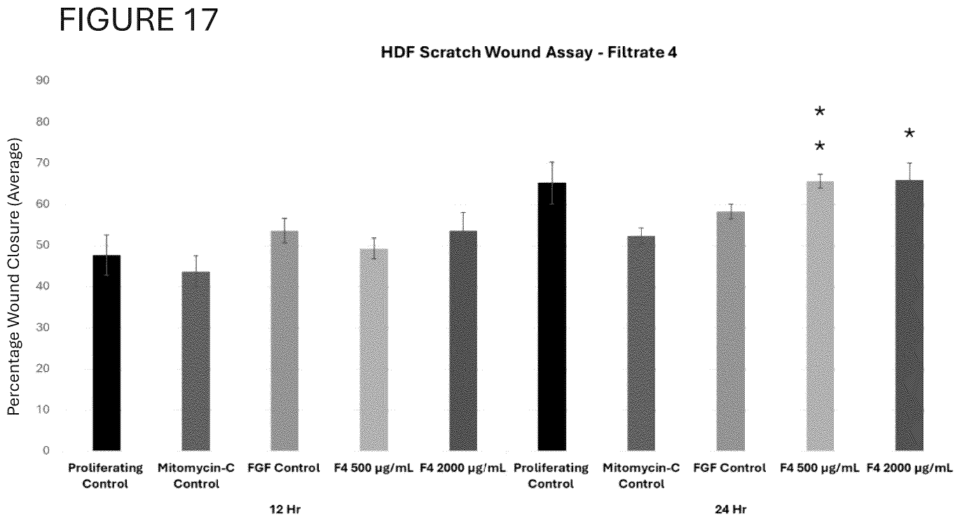

FIG. 17 is a histogram illustrating the outcomes of a scratch wound healing assay conducted on HDF. The experimental groups included: HDF untreated (proliferating control), treated with mitomycin-C only (mitomycin-C control), inhibited with mitomycin-C and treated with Fibroblast Growth Factor (FGF/positive control), or treated with mitomycin-C along with varying concentrations (500 μg/mL or 2000 μg/mL) of filtrate 4. Each experimental condition was performed in triplicates, with two measurements taken per replicate (on both left and right sides of the wound), resulting in a total sample size of n=6. The percentage wound area, determined using the ImageJ wound healing plugin, at the 0-hour timepoint served as the baseline for normalizing subsequent measurements at 12 and 24-hour timepoints for each experimental group. The data is represented as the mean percentage wound area with corresponding SEMs. A t-test assuming unequal variances was conducted, comparing each experimental group to the appropriate mitomycin-C control at a significance level of 5%. Significance levels are denoted by asterisks, with * indicating p<0.05, ** indicating p<0.01, and *** indicating p<0.001; and

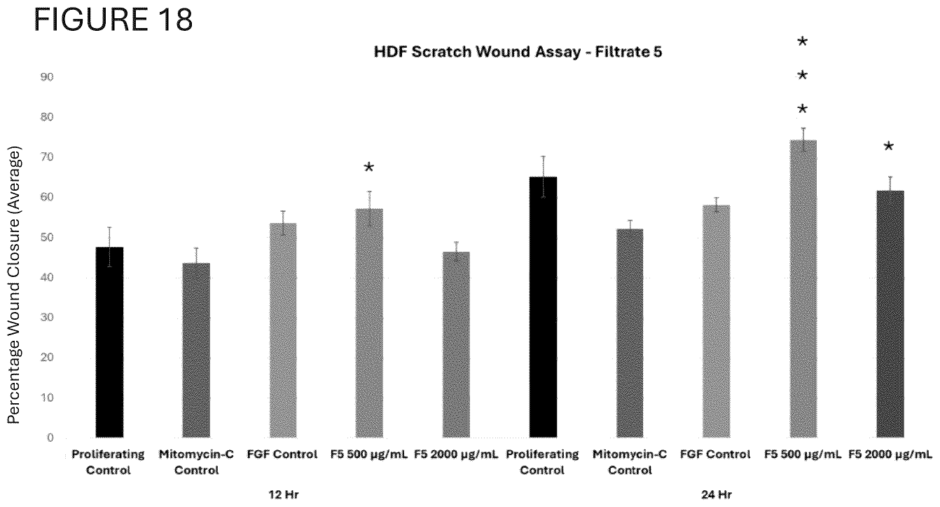

FIG. 18 is a histogram illustrating the outcomes of a scratch wound healing assay conducted on HDF. The experimental groups included: HDF untreated (proliferating control), treated with mitomycin-C(mitomycin-C control), inhibited with mitomycin-C and treated with Fibroblast Growth Factor (FGF/positive control), or treated with mitomycin-C along with varying concentrations (500 μg/mL or 2000 μg/mL) of filtrate 5. Each experimental condition was performed in triplicates, with two measurements taken per replicate (on both left and right sides of the wound), resulting in a total sample size of n=6. The percentage wound area determined using the ImageJ wound healing plugin, at the 0-hour timepoint served as the baseline for normalizing subsequent measurements at 12 and 24-hour timepoints for each experimental group. The data is represented as the mean percentage wound area with corresponding SEMs. A t-test assuming unequal variances was conducted, comparing each experimental group to the appropriate mitomycin-C control at a significance level of 5%. Significance levels are denoted by asterisks, with * indicating p<0.05, ** indicating p<0.01, and *** indicating p<0.001.

TABLE 1

| | | | | | Predicted |
|---|---|---|---|---|---|
| | | Percentage | Percentage | | extract |
| | | Oil | oil | % Protein | protein |
| Rapeseed | Rapeseed | content | content | content | content |
| Cake % | Meal % | predicted | actual | predicted | ug/mL |
| 0 | 100 | 1.91 | 1.91 | 34.055 | 2775 |
| 10 | 90 | 3.894 | 4.29 | 33.345 | 3130 |
| 20 | 80 | 5.878 | 6.68 | 32.636 | 3531 |
| 30 | 70 | 7.862 | 9.00 | 31.926 | 3982 |
| 40 | 60 | 9.846 | 11.32 | 31.216 | 4492 |
| 50 | 50 | 11.83 | 12.62 | 30.507 | 6642 |
| 60 | 40 | 13.814 | 13.92 | 29.797 | 7492 |
| 70 | 30 | 15.798 | 15.78 | 29.087 | 8451 |
| 80 | 20 | 17.782 | 17.64 | 28.377 | 9532 |
| 90 | 10 | 19.766 | 19.695 | 27.668 | 10751 |
| 100 | 0 | 21.75 | 21.75 | 26,958 | 10407 |

### Ratio of Feedstock:Cake/Meal

| Percentage oil | | | | | Standard |
|---|---|---|---|---|---|
| content (NMR) | 1 | 2 | 3 | Average | deviation |
| 20:80 - C:M | 6.05 | 7.14 | 6.84 | 6.68 | 0.460 |
| 40:60 - C:M | 11.5 | 10.99 | 11.47 | 11.32 | 0.234 |
| 60:40 - C:M | 14.17 | 13.87 | 13.72 | 13.92 | 0.187 |
| 80:20 - C:M | 18.14 | 17.05 | 17.73 | 17.64 | 0.450 |

### N Dumas Data

| | | Solid content | | | | | |
|---|---|---|---|---|---|---|---|
| | Protein | UF | Liquid | | | | Protein |
| | Content | Commissioning | mass | kg | kg | g | concentration |
| Sample ID | % | (%) | (kg) | solid | protein | protein | g/L |
| Blend 10% | 37.56 | 2.185 | 0.5 | 0.01092 | 0.004 | 4.1 | 8.21 |
| cake 90% | | | | | | | |
| meal Control | | | | | | | |
| Extraction - | | | | | | | |
| 20 Mar. 2023 | | | | | | | |
| 100% meal | 26.66 | 2.517 | 0.5 | 0.01259 | 0.003 | 3.4 | 6.71 |
| control | | | | | | | |
| Extraction - | | | | | | | |
| 20 Mar. 2023 | | | | | | | |

### OSR Cake Re-Extraction

| | | | | Final |
|---|---|---|---|---|
| | | | | concen- |
| | | Concen- | Dilution | tration |
| Sample name | Absorbance | tration | factor | ug/mL |
| Emulsion 1st extract - | 1.3704 | 1542 | 6 | 9252 |
| 1 in 6 - 1 | | | | |
| Emulsion 1st extract - | 1.3936 | 1581 | 6 | 9484 |
| 1 in 6 - 2 | | | | |
| Emulsion 1st extract - | 1.3810 | 1560 | 6 | 9358 |
| 1 in 6 - 3 | | | | |
| Emulsion 1st extract - | 1.283 | 1397 | 8 | 11175 |
| 1 in 8 - 1 | | | | |
| Emulsion 1st extract - | 1.256 | 1352 | 8 | 10815 |
| 1 in 8 - 2 | | | | |
| Emulsion 1st extract - | 1.2257 | 1301 | 8 | 10407 |
| 1 in 8 - 3 | | | | |
| Emulsion 1st extract - | 1.1651 | 1200 | 10 | 11998 |
| 1 in 10 - 1 | | | | |
| Emulsion 1st extract - | 1.1866 | 1236 | 10 | 12357 |
| 1 in 10 - 2 | | | | |
| Emulsion 1st extract - | 1.1642 | 1198 | 10 | 11983 |
| 1 in 10 - 3 | | | | |
| Emulsion 2nd extract - | 0.8465 | 669 | 6 | 4013 |
| 1 in 6 - 1 | | | | |
| Emulsion 2nd extract - | 0.8389 | 656 | 6 | 3937 |
| 1 in 6 - 2 | | | | |
| Emulsion 2nd extract - | 0.8506 | 676 | 6 | 4054 |
| 1 in 6 - 3 | | | | |
| Emulsion 2nd extract - | 0.7727 | 546 | 8 | 4367 |
| 1 in 8 - 1 | | | | |
| Emulsion 2nd extract - | 0.7781 | 555 | 8 | 4439 |
| 1 in 8 - 2 | | | | |
| Emulsion 2nd extract - | 0.7669 | 536 | 8 | 4289 |
| 1 in 8 - 3 | | | | |
| Emulsion 2nd extract - | 0.7165 | 452 | 10 | 4522 |
| 1 in 10 - 1 | | | | |
| Emulsion 2nd extract - | 0.7382 | 488 | 10 | 4883 |
| 1 in 10 - 2 | | | | |
| Emulsion 2nd extract - | 0.717 | 453 | 10 | 4530 |
| 1 in 10 - 3 | | | | |
| Emulsion 3rd extract - | 0.6961 | 418 | 6 | 2509 |
| 1 in 6 - 1 | | | | |
| Emulsion 3rd extract - | 0.7094 | 440 | 6 | 2642 |
| 1 in 6 - 2 | | | | |
| Emulsion 3rd extract - | 0.7101 | 442 | 6 | 2649 |
| 1 in 6 - 3 | | | | |
| Emulsion 3rd extract - | 0.6441 | 332 | 8 | 2652 |
| 1 in 8 - 1 | | | | |
| Emulsion 3rd extract - | 0.6374 | 320 | 8 | 2563 |
| 1 in 8 - 2 | | | | |
| Emulsion 3rd extract - | 0.6366 | 319 | 8 | 2552 |
| 1 in 8 - 3 | | | | |
| Emulsion 3rd extract - | 0.6019 | 261 | 10 | 2612 |
| 1 in 10 - 1 | | | | |
| Emulsion 3rd extract - | 0.6123 | 279 | 10 | 2785 |
| 1 in 10 - 2 | | | | |
| Emulsion 3rd extract - | 0.6106 | 276 | 10 | 2757 |
| 1 in 10 - 3 | | | | |

| | Average | Percentage reduction |
|---|---|---|
| | concentration ug/mL | from first extraction |
| Emulsion 1st extract | 12113 | |
| Emulsion 2nd extract | 4337 | 36 |
| Emulsion 3rd extract | 2636 | 22 |

### OSR Meal Re-Extraction

| | | | | Final |
|---|---|---|---|---|
| | | | | concen- |
| | | Concen- | Dilution | tration |
| Sample name | Absorbance | tration | factor | ug/mL |
| 1st extraction - 1 in 6 - 1 | 0.8041 | 486.00 | 6 | 2916.00 |
| 1st extraction - 1 in 6 - 2 | 0.8122 | 499.50 | 6 | 2997.00 |
| 1st extraction - 1 in 6 - 3 | 0.8054 | 488.17 | 6 | 2929.00 |
| 1st extraction - 1 in 8 - 1 | 0.724 | 353.17 | 8 | 2825.33 |
| 1st extraction - 1 in 8 - 2 | 0.735 | 370.17 | 8 | 2961.33 |
| 1st extraction - 1 in 8 - 3 | 0.7222 | 349.50 | 8 | 2796.00 |
| 1st extraction - 1 in 10 - 1 | 0.6930 | 300.83 | 10 | 3008.33 |
| 1st extraction - 1 in 10 - 2 | 0.6984 | 309.83 | 10 | 3098.33 |
| 1st extraction - 1 in 10 - 3 | 0.6834 | 284.83 | 10 | 2848.33 |
| 2nd extraction - 1 in 6 - 1 | 0.6510 | 230.83 | 6 | 1385.00 |
| 2nd extraction - 1 in 6 - 2 | 0.6581 | 242.67 | 6 | 1456.00 |
| 2nd extraction - 1 in 6 - 3 | 0.6534 | 234.83 | 6 | 1409.00 |
| 2nd extraction - 1 in 8 - 1 | 0.6246 | 186.83 | 8 | 1494.67 |
| 2nd extraction - 1 in 8 - 2 | 0.6282 | 192.83 | 8 | 1542.67 |
| 2nd extraction - 1 in 8 - 3 | 0.6284 | 193.17 | 8 | 1545.33 |
| 2nd extraction - 1 in 10 - 1 | 0.592 | 132.50 | 10 | 1325.00 |
| 2nd extraction - 1 in 10 - 2 | 0.6097 | 162.00 | 10 | 1620.00 |
| 2nd extraction - 1 in 10 - 3 | 0.5987 | 143.67 | 10 | 1436.67 |
| 3rd extraction - 1 in 6 - 1 | 0.5907 | 130.33 | 6 | 782.00 |
| 3rd extraction - 1 in 6 - 2 | 0.5881 | 126.00 | 6 | 756.00 |
| 3rd extraction - 1 in 6 - 3 | 0.6006 | 146.83 | 6 | 881.00 |
| 3rd extraction - 1 in 8 - 1 | 0.5751 | 104.33 | 8 | 834.67 |
| 3rd extraction - 1 in 8 - 2 | 0.573 | 100.83 | 8 | 806.67 |
| 3rd extraction - 1 in 8 - 3 | 0.5733 | 101.33 | 8 | 810.67 |
| 3rd extraction - 1 in 10 - 1 | 0.5625 | 83.33 | 10 | 833.33 |
| 3rd extraction - 1 in 10 - 2 | 0.5639 | 85.67 | 10 | 856.67 |
| 3rd extraction - 1 in 10 - 3 | 0.5639 | 85.67 | 10 | 856.67 |

| | Average | Percentage reduction |
|---|---|---|
| | concentration ug/mL | from first extraction |
| Extraction 1 | 2931 | |
| Extraction 2 | 1468 | 50.09 |
| Extraction 3 | 824 | 28.12 |

### Materials and Methods

### Preparation of Hydrolysate

Rapeseed meal (RSM) or partially defatted rapeseed cake (RSC) is suspended and contacted with water adjusted to a pH of around 10-11 (using NaOH), by means of high-shear mixing (Silverston-type mixer), until well incorporated. A solids:liquid ratio of 1:10 is used nominally. The resulting slurry is mixed in a stirred in a vessel for around 40 minutes, with an addition of sodium sulphite 0.025% w/v and sodium hydroxide used to maintain the pH of around 10-11.

The extracted solids are removed by either centrifugation or filtration to leave a clarified supernatant or filtrate containing solubilised protein.

The collected solids may be further extracted, by mixing in the stirred vessel, with a second volume of pH-adjusted water, maintaining a suitable solid:liquid volume ratio (e.g. 1:10). The supernatants or filtrates from the first and subsequent extractions are pooled. The pooled supernatants undergo a concentration/diafiltration stage using ultrafiltration membrane of either 10, 3 or 1 kDa NMWCO polyethersulfone (PES). The concentrate is maintained at around pH 8-10 using NaOH. This stage yields a retentate of reduced volume and a filtrate. The filtrate may be (partially) reused used for extraction of the RSM above.

The retentate (from the concentration/diafiltration stage) then undergoes enzyme hydrolysis with either immobilised or free enzyme(s), in a stirred reactor vessel. The enzyme(s) are either mixtures of endoproteases (preferred) and optionally exoproteases depending on the desired product. Examples include Alcalase® 2.4 L (subtilisin) or Protamex®, as endoproteases. A suitable exoprotease includes Flavourzyme®. Typically addition rates of free enzyme and hydrolysis conditions are given below.

| | Supplier stated | Nominal addition | Temperature |
|---|---|---|---|
| Enzyme | specific | per 100 L | (° C.)/pH |
| example | activity (U/g) | hydrolysis (U) | working range |
| Alcalase ® | >2.4 | 1.2 | 30-65° C. |
| 2.4L | | | pH 6.5-10 |
| (subtilisin) | | | |
| Protamex ® | >1.5 | 0.75 | 55-60° C. |
| | | | pH 6-9 |
| Flavourzyme ® | >500 | 250 | 30-65° C. |
| | | | pH 4.5-7.5 |

The temperature and pH of the hydrolysis are maintained at, or near, the optima for the enzymes in use, nominally 50° C. and pH 8.5 respectively. The hydrolysis is performed for around 5 hours or until the molecular weight profile of the hydrolysis product (for example, determined by HPLC-size exclusion chromatography) reaches a constant or limit.

In the case of immobilised enzymes, suitable immobilisation onto a beaded polymethacrylate (e.g. Purolite Lifetech™ ECR8415F) support can be achieved using covalent chemistry (e.g. amino-linkages or alternatively via epoxy-linkages). Alternative immobilisation substrates include e.g. Resindion and Sepabeads. This mode of hydrolysis operation can be performed in a plug-in rotating bed reactor, with the immobilised enzyme(s) maintained within the rotating bed basket, or alternatively using a packed or fluidised bed reactor.

The resulting hydrolysate is then further filtered (ultrafiltration) using either a 1 kDa or 3 kDa NMWCO PES membrane, with a typical split of 80:20 filtrate to retentate. The filtrate contains the soluble peptides of interest, and the retentate is either sent to waste or recycled to an earlier stage of the process, to allow further hydrolysis of protein to peptides to increase recovery in the product.

The material was freeze-dried to yield a solid peptide product, with a nominal protein content by N-analysis (Dumas) of 9.3%.

### Downstream Processing of the Peptide-Containing Filtrate

An ion exchange (IE) column can be used to further concentrate the peptides, possibly separating into positively and negatively charged peptides depending on specific application is found which requires selective separation. An aim of this step is to enrich the protein content of the peptide solution product from the UF stage.

The concentrated hydrolysis product is then either passed through a sterile filter (0.2 or 0.45 μm) and sent for final storage if a liquid product is preferred or sent to be dried to provide a powder product using either freeze or spray drying.

### Cell Culture

The CnT human juvenile epidermal keratinocyte progenitors (pooled) (HPEKp) were cultured in the absence of calcium in CnT-PR media (CnT-PR, CELLnTECH) supplemented with 1% Pen-Strep and cultured as per manufacturer instructions (HPEKp, CELLnTECH). Cells were maintained in log phase growth, incubated in 5% CO₂ at 37° C., and the growth media was changed every two days.

### Proteomic Analysis

Filtrate 1 peptides were dissolved in buffer A (0.1% TFA/Milli-Q water) and purified using STAGE tipping (Agilent Bond Omix C18 manual). Briefly, C18-StageTips (3M™ Empore™ C18 SPE Disks) was first conditioned with 50 μl MeOH and then equilibrated with 50 μl buffer B (0.1% TFA/acetonitrile) and 50 μl buffer A. The peptides were loaded onto the primed tips and then washed in 50 μl buffer A before eluting in 40 μl buffer B. The sample was concentrated in a vacuum concentrator, resuspended in 40 μl buffer A, and then sonicated for 1 minute. Liquid-chromatography tandem mass-spectrometry was performed on the processed peptides with LTQ Orbitrap Velos Pro (Thermo Fisher Scientific, Bremen, Germany). From MS data, protein identification was achieved with the Andromeda peptide database provided via MaxQuant (v2.1.0.0). Filtrate 1 parent proteins were considered by DAVID for functional annotations clustering analysis.

2,5-diphenyl-2H-tetrazolium-bromide (MTT) Cytotoxicity Assay

The cytotoxicity profiles of rapeseed filtrate 1 extracts in HPEKp, HDF, and NSCLC cells were determined using the MTT assay (Thiazolyl Blue Tetrazolium Bromide, Sigma-Aldrich®, Catalogue no. M5655). Cells were harvested, washed twice with phosphate-buffered saline (PBS), and resuspended in media. A total of 1.5×10\hat{ }4 cells in a volume of 100 μl were plated per well in 96-well plates and incubated at 37° C. for 24 hours to ensure correct cell adhesion. The cell suspension was then replaced with 100 μl of fresh media containing either no bioactive peptides or a sequential two-fold serial dilution of filtrate 1 peptides ranging from 62.5-2000 μg/mL and incubated for 24 and 48 hours. Following drug incubation, the media was removed, and the wells were washed with PBS to remove any excess background caused by rapeseed extract. A total of 100 μl of media containing MTT (5 mg/mL) was added to wells and incubated for 3 hours. The media was then replaced with 100 μl of DMSO, and the plate was gently shaken for 15 minutes to ensure complete solubilisation of the formazan crystals. Absorbance was measured at 560 nm, and the background was subtracted at 670 nm using a spectrophotometer. Each MTT plate included 6 replicates. A one-way ANOVA analysis was performed on the absorbance data using Microsoft Excel at a significance level of 5%. The cytotoxicity profiles of rapeseed filtrate 1 extracts were determined by comparing the absorbance values of cells treated with the filtrate 1 extracts to those of untreated cells.

### Transmigration Assay

The chemoattractant capacity of rapeseed filtrate 1 hydrolysates on HPEKp and HDF cells was evaluated using the QCM Chemotaxis Cell Migration Assay (Sigma-Aldrich®, Catalogue no. ECM508). Briefly, 100 μl of 25 μg/mL collagen 1 was added to the upper well inserts and incubated overnight at room temperature to generate a matrix for cell attachment. The inserts were then washed twice with PBS to remove any remaining unbound collagen. A chemoattractant-free cell suspension containing 1.5×10\hat{ }5 cells in 300 μl was added to the upper inserts. A 24-well plate was prepared by adding 500 μl of growth media containing no FBS or filtrate 1 hydrolysates (untreated control), 5% FBS and no filtrate 1 hydrolysates, or 5% FBS with 1000 μg/mL of filtrate 1 hydrolysates to each well. The upper inserts containing cells were placed into the wells containing the cell-free media and incubated in 5% CO₂ at 37° C. overnight. The remaining media with the unmigrated cells was removed, and the upper inserts were stained with 400 μl of crystal violet for 20 minutes at room temperature and then washed several times in dd.H2O. The unmigrated cells remaining on the topside of the upper insert interior were carefully removed by swabbing to reduce potential background. The cleaned stained insert was placed into a fresh well and 200 μl of extraction buffer was added and incubated for 15 minutes at room temperature. Finally, 100 μl of the stained dye solution was added to a fresh 96-well plate and the optical density was read with a spectrophotometer at 560 nm.

### Cell Culture Human Keratinocytes

Human (HaCaT) keratinocytes were purchased from CLS Cell Lines Service GmbH and cultured in Dulbecco's Modified Eagle Medium (DMEM) supplemented with 10% foetal bovine serum, 4.5 g/L glucose, and 4 mM L-Glutamine. The cells were maintained at 37° C. in a humidified incubator containing 5% CO₂ and sub-cultured at a ratio of 1:5.

### Cell Viability (MTS) Assay

HaCaT cells were seeded out in a 96-well plate at 2.5×10⁵ cells/mL and cultured until 60% confluence. Thereafter, cells were treated with hydrolysate at concentrations of 62.5 and 125 μg/mL. Hydrocortisone (10 nM) was used as reference drug. One hour later, cells were stimulated with recombinant TNF-α (10 ng/ml) for a further 24 hours.

At the end of the experiment, 100 μL of culture medium was removed followed by addition of 20 μL of CellTiter 96® AQueous One (MTS) Solution (Promega) into each well of the 96-well plate containing 100 μL of culture medium. The plate was then incubated in 5% CO₂ incubator at 37° C. for 4 h. Then, absorbance was read at 490 nm using Tecan M Nano microplate reader.

### Effect of Hydrolysate on TNF-α and IL-6 Production in HaCaT Keratinocytes

HaCaT cells were seeded out at 2.5×10⁵ cells/mL and cultured until 60% confluence. Thereafter, cells were treated with hydrolysate at concentrations of 62.5 and 125 μg/mL. Hydrocortisone (10 nM) was used as reference drug. One hour later, cells were stimulated with recombinant TNF-α (10 ng/ml) for a further 24 hours. Thereafter, culture medium was collected and centrifuged to obtain supernatants.

Culture supernatants were then analysed for levels of pro-inflammatory cytokines TNF-α and IL-6 using human ELISA kits (Thermo Scientific), according to the manufacturer's instruction. Absorbance was read at 450 nm using Tecan M Nano microplate reader, and concentrations of TNF-α and IL-6 released were determined from a standard curve.

### EXAMPLES

### Example 1

The mean amino acid composition and the distribution of amino acid subtypes according to mass spectrometry analysis of filtrate 1 peptides are depicted in FIG. 1. Glutamine (21%), glycine (10%), arginine (8%), valine (6%), and proline (6%) are the top five constituent amino acids identified from a cohort of 995 peptides mapped by proteomic analysis (FIG. 1A). Notably filtrate 1 peptides contained no selenocysteine or pyrrolysine and only 0.018% cysteine and 0.355% tryptophan. Non-polar regions (42%) and polar regions (38%) constitute the vast majority of filtrate 1 peptides, whilst acidic (15%) and basic amino acids (5%) appear to be less abundant (FIG. 1B).

### Example 2

FIG. 2 illustrates the span and mass of filtrate 1 peptides as observed from mass spectra. Based on the 995 identifiable peptides, the mean amino acid chain length is 10 amino acids long, comprising 15.48% of sample peptides (FIG. 2A). The majority (51.48%) of filtrate 1 peptides appear to span between 9-12 amino acids, with just 4.92% of peptides exceeding a chain length of 19 amino acids. The mean peptide molecular weight observed has a range of 1079.492 to 1219.492 (18.89%), with 47.54% of sample peptide molecular weights identified as being between 939.492 and 1359.492 (FIG. 2B). Notably, just 5.33% of filtrate 1 peptides exceeded a molecular weight of 2199.492.

### Example 3

In silico processing of the 995 filtrate 1 peptides resulted in the identification of 78 unique proteins. FIG. 3 represents the DAVID functional annotations bioinformatic analysis conducted on 73/78 mapped Brassica napus proteins. DAVID's gene ontology clustering feature highlighted molecular functions of the established filtrate 1 proteins as DNA-binding (27 protein ID's), seed storage proteins (12 protein ID's), storage proteins (12 protein ID's), or antioxidants (2 protein ID's) (FIG. 3A). The cellular localisation of filtrate 1 proteins appears to be primarily mapped to the regions of the chromosome (27 protein ID's) or nucleus (27 protein ID's) and to a lesser extent lipid droplets (4 protein ID's) (FIG. 3B). The clustering analysis exclusively identified protein biosynthesis as a biological process associated with the observed protein dataset characterised by 3 protein IDs, including (rape) hypothetical protein (BnaC04g13200D), protein (BnaAnng15460D), and protein (BnaC08g43440D) (FIG. 3C).

### Example 4

A histogram displaying the 24 (A) and 48-hour (B) MTT results of primary human epidermal keratinocytes pooled (HPEKp) treated with CnT-PR dissolved filtrate 1 extracts at concentrations ranging from 0 to 2000 μg/mL; see FIG. 4. The mean percentage viability values, calculated by comparing the absorbance of treated to untreated HPEKp cells, are plotted on the chart. Error bars indicate the standard error of the mean (SEM).

### Example 5

A histogram displaying the 24 (A) and 48-hour (B) MTT results of human dermal fibroblasts (HDF) cells treated with fibroblast growth media dissolved filtrate 1 extracts at concentrations ranging from 0 to 2000 μg/mL; see FIG. 5. The mean percentage viability values and SEM are plotted.

### Example 6

A histogram displaying the 24 (A) and 48-hour (B) MTT results of A549 cells treated with Dulbecco's MEM dissolved filtrate 1 extracts at concentrations ranging from 0 to 2000 μg/mL; see FIG. 6. The mean percentage viability values and SEM are plotted.

### Example 7

A histogram depicting the results of the transmigration assay conducted on HPEKp cells; see FIG. 7. The percentage increase in cell migration over 24 hours, with the control (absence of a chemoattractant) and two experimental conditions: HPEKp cells treated with fetal bovine serum (FBS) and HPEKp cells treated with FBS supplemented with 1000 μg/mL of filtrate 1 extract in CNT-PR media are displayed. The values plotted represent the mean cell migration of two experimental conditions compared to untreated samples, and the error bars indicate the SEM.

A histogram depicting the results of the transmigration assay conducted on HDF cells; see FIG. 8. The percentage increase in cell migration over 24 hours, with the control (absence of a chemoattractant) and two experimental conditions: HDF cells treated with FBS and HDF cells treated with FBS supplemented with 1000 μg/mL of filtrate 1 extract in HDF growth media are displayed. The values plotted represent the mean cell migration of two experimental conditions compared to untreated samples, and the error bars indicate the SEM.

### Example 8

Table 1 shows that the protein contents of the liquid fraction resulting from extractions using a 100% meal feedstock and a 90% Meal-10% Cake ‘Blended’ feedstock are 6.71 g/L and 8.21 g/L respectively. This indicates that using a blend at this ratio of cake to meal in the extraction step leads to a 22% increase in protein (N Dumas, Table 1b) yield in the resulting extract. Increasing the ratio of cake to meal shows a consistent relative increase to final yield of protein in the extract.

### Example 9

### Protease Extraction

In order to increase protein yield and to streamline the overall process by reducing the number of steps required, an alteration to the process has been implemented-protease extraction. This involves the addition of proteases to the high shear mixed slurry which is kept at a pH of 8.00 and a temperature of 50° C. These are the conditions used for the hydrolysis step which is being brought forward and replacing the extraction stirring step of bringing the pH up to 10.5 and maintaining that for 1 hour of stirring. By merging the extraction and hydrolysis steps, the overall number of process stages is reduced as well as the total amount of sodium hydroxide required.

Besides increased efficiency and reduced cost of sodium hydroxide and the transporting/stirring of liquid, the primary benefit of protease extraction is the increase in yield of solubilized protein from the OSR solid into the liquid fraction. The following analysis by Martindale analytical using Nitrogen by Dumas shows the increase in nitrogen in the liquid fraction following solid-liquid separation. This relates directly to solubilized protein. The percentage protein column shows an increase from 26.5% to 42.8% on average. This is a 61% relative increase in protein yield from the original methodology.

| | | | | | Freeze | | | |
|---|---|---|---|---|---|---|---|---|
| Process | | Vial | | Sample | dried | Solids | | % |
| conditions | Samples | TARE | Vial full | weight | vial | (g) | % solids | protein |
| Standard | UF Filt | 8.6935 | 12.7039 | 4.0104 | 8.8000 | 0.1065 | 2.66 | 27.8 |
| | 3 hr - | | | | | | | |
| | 1 Aug. 2023 | | | | | | | |
| Standard | UF Filt | 8.5578 | 12.5218 | 3.9640 | 8.6447 | 0.0869 | 2.19 | 25.2 |
| | 30 m - | | | | | | | |
| | 25 Sep. 2023 | | | | | | | |
| Protease | UF Filt | 9.8972 | 13.9273 | 4.0301 | 10.0288 | 0.1316 | 3.27 | 43.7 |
| extraction | 1 hr 30 - | | | | | | | |
| | 20 Nov. 2023 | | | | | | | |
| Protease | UF Filt | 8.5811 | 12.5963 | 4.0152 | 8.7088 | 0.1277 | 3.18 | 41.9 |
| extraction | 1 hr 30 - | | | | | | | |
| | 27 Nov. 2023 | | | | | | | |

### Example 10

### Actinidin Protease

An enzyme sourced from kiwi fruit which is used to hydrolyze protein primarily as a meat tenderizer has been trialed on our OSR feedstock. Trials have been carried out on the 10 L scale to obtain UF filtrate from an Actinidin protease extraction of the feedstock. These samples undergo analysis to determine what kind of peptide profile is produced compared to the standard Subtilisin-Protamex hydrolysis as a control. Earlier trials on the 1-liter scale proved that the enzyme does have activity when hydrolyzing OSR extract.

### Example 11

### Effect of Hydrolysate on the Viability of HaCaT Cells

Results of cell viability experiments in FIG. 12 show that treatment of HaCaT cells with 62.5 and 125 g/mL of the hydrolysate resulted in significant (p<0.001) and concentration-dependent increase in the number of viable cells. These results suggest that the hydrolysate increases the proliferation of these cells, in vitro.

### Example 12

### Effects of Hydrolysate on TNF-α-Induced Increased Production of TNF-α and IL-6 in HaCaT Keratinocytes

Following stimulation of HaCaT cells with recombinant TNF-α (10 ng/ml), production of TNF-α significantly increased from ~8.7 μg/mL to ~760 μg/mL. Pre-treatment with 62.5 μg/mL of the hydrolysate resulted in significant (p<0.0001) reduction in TNF-α production to 72.1% when compared with stimulated cells. On increasing the concentration of the hydrolysate to 125 μg/mL, TNF-α production was further reduced to 47.6% while it was reduced to 27.5% in cells pre-treated with hydrocortisone (10 nM) (FIG. 13).

### Example 13

In separate experiments, supernatants obtained from TNF-α-stimulated HaCaT cells showed significant (p<0.0001) increase in levels of IL-6. However, when cells were treated with 62.5 and 125 125 μg/mL of the hydrolysate, concentrations of IL-6 in culture supernatants were reduced to 68.8% and 52%, respectively in comparison to TNF-α-stimulated HaCaT cells (FIG. 14).

### Example 14

Benchtop scale trials have been undertaken which remove the addition of sodium sulfite from the process. Originally added as a preventative measure against oxidation of proteins, the removal of sodium sulfite was not found to have an impact on extracted protein, rate or degree of hydrolysis. Pilot plant scale trials have been undertaken with sodium sulfite removed from the process without clear drawbacks. The removal of this input material will reduce overall cost and improve process efficiency.

### Example 15

In a series of scratch wound assays the degree of wound area percentage closure after 12 hours and 24 hours was assessed by taking two measurements per sample each in triplicate (n=6). The material ‘oilseed rape hydrolysate Filtrate 2’ (Original OSR hydrolysate (second pass)) tested in human dermal fibroblasts (HDF) was compared to 1) HDF untreated (proliferating control), 2) inhibited with mitomycin-C only (negative control), and 3) both inhibited with mitomycin-C and stimulated with Fibroblast Growth Factor (positive control). The F2 filtrate displayed after 24 h a statistically significant (p<0.05) percentage wound closure at both 500 μg/mL and 2000 μg/mL concentrations when compared to the controls (FIG. 15)

### Example 16

Next in the series of scratch wound assays the degree of wound area percentage closure after 12 and 24 hours was assessed by taking two measurements per sample each in triplicate (in=6). The material ‘oilseed rape hydrolysate Filtrate 3’ (Standard conditions—protease extraction using subtilisin and protamex) tested in human dermal fibroblasts (HDF) as compared to 1) HDF untreated (proliferating control), 2) inhibited with mitomycin-C only (negative control), and 3) both inhibited with mitomycin-C and stimulated with Fibroblast Growth Factor (positive control). The F3 filtrate displayed even greater statistical significance (p<0.01) percentage wound closure at both 500 μg/mL and 2000 μg/mL concentrations (FIG. 16).

### Example 17

Furthermore, in the series of scratch wound assays the degree of wound area percentage closure after 12 and 24 hours was assessed by taking two measurements per sample each in triplicate (n=6). The material ‘oilseed rape hydrolysate Filtrate 4’ (NF filtrate diluent—protease extraction using subtilisin and protamex in recycled NF filtrate diluent) tested in human dermal fibroblasts (HDF) as compared to 1) HDF untreated (proliferating control), 2) inhibited with mitomycin-C only (negative control), and 3) both inhibited with mitomycin-C and stimulated with Fibroblast Growth Factor (positive control). The F4 filtrate displayed high statistical significance (p<0.01) percentage wound closure at the 500 μg/mL concentration and slightly lower statistical significance (p<0.05) at the 2000 μg/mL concentration (FIG. 17)

### Example 18

Concluding the series of scratch wound assays the degree of wound area percentage closure after 12 and 24 hours was assessed by taking two measurements per sample each in triplicate (n=6). The material ‘oilseed rape hydrolysate Filtrate 5’ (Actinidin—protease extraction using actinidin) tested in human dermal fibroblasts (HDF) as compared to 1) HDF untreated (proliferating control), 2) inhibited with mitomycin-C only (negative control), and 3) both inhibited with mitomycin-C and stimulated with Fibroblast Growth Factor (positive control). The F5 filtrate displayed extremely high statistical significance (p<0.001) percentage wound closure at the 500 μg/mL concentration and a lower statistical significance (p<0.05) at the 2000 μg/mL concentration (FIG. 18).

### REFERENCES

Alam, H., Sehgal, L., Kundu, S. T., Dalal, S. N., and Vaidya, M. M., 2011. Novel function of keratins 5 and 14 in proliferation and differentiation of stratified epithelial cells. Molecular biology of the cell, 22 (21), pp. 4068-4078.
Amigo, L., and Hernández-Ledesma, B., 2020. Current Evidence on the Bioavailability of Food Bioactive Peptides. Molecules (Basel, Switzerland), 25 (19), pp. 4479.
Chakrabarti, S., Guha, S., and Majumder, K., 2018. Food-Derived Bioactive Peptides in Human Health: Challenges and Opportunities. Nutrients, 10 (11), pp. 1738.
de Paz-Lugo, P., Lupiáñez, J. A., and Meléndez-Hevia, E., 2018. High glycine concentration increases collagen synthesis by articular chondrocytes in vitro: acute glycine deficiency could be an important cause of osteoarthritis. Amino acids, 50 (10), pp. 1357-1365.
European Commission (Committee for the Common Organisation of Agricultural Markets), 2022. Oilseeds and Protein Crops market situation. Available at: https://circabc.europa.eu/sd/a/215a681a-5f50-4a4b-a953-e8fc6336819c/oilseeds-marketsituation.pdf (Accessed: 14/09/22).
Ferrero, R. L., Soto-Maldonado, C., Weinstein-Oppenheimer, C., Cabrera-Muñoz, Z., and Zúñiga-Hansen, M. E., 2021. Antiproliferative Rapeseed Defatted Meal Protein and Their Hydrolysates on MCF-7 Breast Cancer Cells and Human Fibroblasts. Foods (Basel, Switzerland), 10 (2), pp. 309.
Ganceviciene R, Liakou A I, Theodoridis A, Makrantonaki E, and Zouboulis C C., 2012. Skin anti-aging strategies. Dermatoendocrinol, 4 (3), pp. 308-19.
Gorissen, S., Crombag, J., Senden, J., Waterval, W., Bierau, J., Verdijk, L. B., and van Loon, L., 2018. Protein content and amino acid composition of commercially available plant-based protein isolates. Amino acids, 50 (12), pp. 1685-1695.
Karna, E., Szoka, L., Huynh, T., and Palka, J. A., 2020. Proline-dependent regulation of collagen metabolism. Cellular and molecular life sciences: CMLS, 77 (10), pp. 1911-1918.
Kramer, J. K., Sauer, F. D., Wolynetz, M. S., Farnworth, E. R., and Johnston, K. M., 1992. Effects of dietary saturated fat on erucic acid induced myocardial lipidosis in rats. Lipids, 27 (8), pp. 619-23
Lu, C., Napier, J. A., Clemente, T. E., and Cahoon, E. B., 2011. New frontiers in oilseed biotechnology: meeting the global demand for vegetable oils for food, feed, biofuel, and industrial applications. Current opinion in biotechnology, 22 (2), pp. 252-259.
Raboanatahiry, N., Li, H., Yu, L., and Li, M., 2021. Rapeseed (Brassica napus): Processing, Utilization, and Genetic Improvement. Agronomy, 11 (9), pp. 1776.
Russo, M., Yan, F., Stier, A., Klasen, L., and Honermeier, B., 2021. Erucic acid concentration of rapeseed (Brassica napus L.) oils on the German food retail market. Food science & nutrition, 9 (7), pp. 3664-3672.
Sigma-Aldrich®, no date. MTT Assay Protocol for Cell Viability and Proliferation. Available at: https://www.sigmaaldrich.com/GB/en/technical-documents/protocol/cell-culture-and-cell-culture-analysis/cell-counting-and-health-analysis/cell-proliferation-kit-i-mtt (Assessed: Feb. 9, 2022).
Song, Y., Wu, C., Zhang, X., Bian, W., Liu, N., Yin, S., Yang, M., Luo, M., Tang, J., and Yang, X., 2019. A short peptide potentially promotes the healing of skin wound. Bioscience reports, 39 (3), BSR20181734.
Tadesse, S. A., and Emire, S. A., 2020. Production and processing of antioxidant bioactive peptides: A driving force for the functional food market. Heliyon, 6 (8), e04765.
Uniprot., no date. A0A078JE19 A0A078JE 19_BRANA. Available at: https://www.uniprot.org/uniprotkb/A0A078JE19/entry (Accessed: 22/09/22).
Vera-Aviles, M., Vantana, E., Kardinasari, E., Koh, N. L., and Latunde-Dada, G. O., 2018. Protective Role of Histidine Supplementation Against Oxidative Stress Damage in the Management of Anemia of Chronic Kidney Disease. Pharmaceuticals (Basel, Switzerland), 11 (4), pp. 111.
Williams, J. Z., Abumrad, N., and Barbul, A., 2002. Effect of a specialized amino acid mixture on human collagen deposition. Annals of surgery, 236 (3), pp. 369-375.
Williams, S. P. C., 2015. The power of peptides. Available at: https://www.aocs.org/stay-informed/inform-magazine/featured-articles/the-power-of-peptides-november/december-2015?SSO=True (Accessed: 14/09/22)
Woodfield, H. K., and Harwood, J. L., 2017. Oilseed Crops: Linseed, Rapeseed, Soybean, and Sunflower,

### Editor(s): Brian Thomas, Brian G Murray, Denis J Murphy,

### Encyclopedia of Applied Plant Sciences (Second Edition), Academic Press, 2017, pp. 34-38.

Woźniak, E., Waszkowska, E., Zimny, T., Sowa, S., and Twardowski, T., 2019. The Rapeseed Potential in Poland and Germany in the Context of Production, Legislation, and Intellectual Property Rights. Frontiers in plant science, 10, pp. 1423.
Zaky, A. A., Simal-Gandara, J., Eun, J. B., Shim, J. H., and Abd El-Aty, A. M., 2022. Bioactivities, Applications, Safety, and Health Benefits of Bioactive Peptides From Food and By-Products: A Review. Frontiers in nutrition, 8, pp 815640.

1. A composition comprising a plurality of peptides wherein said peptides are prepared from a plant extract obtained by protease digestion.

2.-3. (canceled)

4. The composition of claim 1, wherein said plurality of peptides comprise peptides that vary in length of between 2 to 25 amino acids.

5. The composition of claim 1, wherein said plant extract is obtained from a plant of the genus Brassica.

6. The composition of claim 1, wherein said plurality of peptides is obtained by digestion with at least two endopeptidases.

7. The composition of claim 6, wherein at least one endopeptidase is substilisin, bacillolysin or substilisin and bacillolysin.

8.-9. (canceled)

10. The composition of claim 1, wherein said protease is actinidin.

11. The composition according to claim 10 wherein actinidin is combined with at least one further protease.

12. The composition according to claim 11 wherein said further protease is substilisin, bacillolysin, or both substilisin and bacillolysin.

13.-14. (canceled)

15. The composition of claim 1, wherein said composition further comprises a second different therapeutic agent.

16. The composition according to claim 15 wherein said second therapeutic agent is an analgesic.

17. The composition of claim 1, wherein said composition comprises traces of sodium or sodium salts.

18. A method of treating a cutaneous or sub-cutaneous condition in a subject, comprising administering an effective amount of the composition of claim 1 to the subject.

19. The method according to claim 18 wherein said cutaneous condition is a cutaneous ulcer.

20. The method according to claim 19 wherein said ulcer is a diabetic ulcer associated with diabetic polyneuropathy.

21. A method of treating a cutaneous or sub-cutaneous wound in a subject, comprising administering an effective amount of the composition of claim 1 to the subject.

22. The method according to claim 21 wherein said cutaneous wound is a burn or scald.

23. A cosmetic treatment method, comprising administering an effective amount of the composition of claim 1 to a subject.

24. A method of treating hypertension or an inflammatory disease or condition in a subject, comprising administering an effective amount of the composition of claim 1 to the subject.

25. (canceled)

26. The method according to claim 24, wherein said inflammatory disease or condition is an autoimmune inflammatory disease or condition.

27. The method according to claim 24, wherein said inflammatory disease or condition is an inflammatory bowel disease.

28. The method according to claim 27 wherein said inflammatory bowel disease is selected from the group consisting of: Crohn's disease, ulcerative colitis, coeliac disease, collagenous colitis, lymphocytic colitis, ischaemic colitis, diversion colitis, and Behcet's colitis.

29.-30. (canceled)

31. A method for preparing a plurality of peptides, comprising:
i) preparing plant material to provide an undigested plant homogenate;
ii) separating the homogenate in i) to solid plant material and soluble plant material;
iii) treating said soluble plant material to provide a concentrated soluble plant extract; and
iv) digesting said concentrated plant extract with one or more proteases;
or comprising:
i) preparing plant material to provide a plant homogenate combined with one or more proteases to form a combined plant/protease homogenate;
ii) incubating the combined homogenate to form a protease digested homogenate; and optionally
iii) separating the protease digested plant homogenate from the undigested plant homogenate.

32. The method according to claim 31 wherein said homogenate of step i) comprises potassium hydroxide.

33.-34. (canceled)

35. The method according to claim 31, wherein said plurality of peptides is obtained by digestion with at least two endopeptidases.

36. The method according to claim 31, wherein at least one endopeptidase is subtilisin, bacillolysin or subtilisin and bacillolysin.

37.-38. (canceled)

39. The method according claim 31, wherein protease digestion includes actinidin.

40. The method according to claim 31, wherein said plant material the ratio of meal to cake is selected from the group: 10:90, 20:80, 30:70, 40:60, 50:50, 60:40, 70:30, 80:20 and 10:90.

41. (canceled)

42. A plurality of peptides obtained by the method according to claim 31.
